(19) United States
(12) Patent Application Publication
(10) Pub. No.: US 2008/0014167 A1
(43) Pub. Date: Jan. 17, 2008

(54) SURFACTANT-CONTAINING CLEANSING COMPOSITION WITH SPECIAL PROTEINS

(76) Inventors: Ingrid Bitter, Krefeld (DE); Dieter Goddinger, Klein Nordende (DE)

Correspondence Address: PAUL & PAUL, 2000 MARKET STREET, PHILADELPHIA, PA, 19103-3229, US

(21) Appl. No.: 11/778,150

(22) Filed: Jul. 16, 2007

Related U.S. Application Data
(63) Continuation of application No. PCT/EP05/12938, filed on Dec. 2, 2005.

(30) Foreign Application Priority Data
Jan. 18, 2005 (DE) 10 2005 002 486.6

(51) Int. Cl. A61K 8/97; A61K 8/64; A61K 36/889; A61K 36/886; A61K 36/42

(52) U.S. Cl. 424/70.14; 424/74; 424/727; 424/758; 424/744

(57) ABSTRACT

A surfactant-containing cleansing composition is comprised of a surfactant and (a) at least one protein from yogurt and (b) one or more substances from the group of plant extracts and/or plant milks and/or vitamins, provitamins and vitamin precursors, and derivatives of vitamins, provitamins and vitamin precursors in the weight ratio a):b) 10:1 to 1:4.

### CROSS-REFERENCE TO RELATED APPLICATIONS.

This application is a continuation under 35 U.S.C. Sections 365 and 120 of International Application No. PCT/EP2005/012938, filed Dec. 2, 2005. This application also claims priority under 35 U.S.C. Section 119 of German Application No. DE 10 2005 002 486.6, filed Jan. 18, 2005.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to surfactant-containing cleansing compositions based on a special active ingredient combination, and to the use of this active ingredient combination for smoothing the skin and for regulating skin moisture.

Cleansing compositions for skin and hair, as are commercially available, for example, as liquid soaps, shampoos, shower baths, foam baths, shower and washing gels, not only have to have good cleaning power, but should also be well tolerated by the skin and the mucosa and not lead to severe degreasing or skin dryness even upon frequent use.

Customary cosmetic cleansing products comprise surfactants in amounts of from about 10 to 15% by weight in order to achieve a satisfactory cleaning performance. Following the application of such a cleansing product, however, the consumer often perceives his or her skin condition as not ideal, which is to be attributed predominantly to the slightly irritating effect of most surfactants. The condition of the skin following application is perceived as dry, taut and sometimes rough, for which reason the subsequent application of creams or lotions is required. This has firstly the disadvantage of higher time expenditure and secondly the disadvantage of higher cost since at least two products are required.

(2) Description of Related Art, Including Information Disclosed Under 37 C.F.R. §§ 1.97 and 1.98.

In the past, skin-treatment compositions have been proposed which comprise yogurt derivatives as skin moisturizers.

EP 518 192 B1 discloses a nature-cosmetic skincare or haircare composition with a content of juices of pressed plants or parts of plants, including lemon juice and olive oil, and water, characterized by a content of parsley juice, celery juice from leaves and tubers, dandelion juice from leaves, juice of peeled kiwis, yogurt, juice of pitted green olives and sea salt. The compositions disclosed here can also comprise yogurt.

EP 315 541 B1 discloses a solid preservative-free skin-treatment composition based on yogurt or kefir, which is mixed with water directly prior to application and makes the skin smooth and soft.

GB 2,037,160 A discloses a product which is obtained from skimmed milk under the conditions of yogurt production by protein hydrolysis and can be used in cosmetic and pharmaceutical compositions as moisturizer.

However, these documents disclose no surfactant-containing cleansing products, and for this reason there continues to be a need for cosmetic cleansing compositions which ensure a rapid, uncomplicated and good cleaning performance in one step without the skin condition being adversely affected thereby, and without subsequent treatment with skincare compositions being required.

Completely surprisingly, a surfactant-containing cleansing composition has now been developed which combines these requirements in an ideal way.

### Brief Summary of the Invention

### BRIEF DESCRIPTION OF THE SEVERAL VIEWS OF THE DRAWING(S)

Not Applicable

### DETAILED DESCRIPTION OF THE INVENTION

The invention therefore provides a surfactant-containing cleansing composition comprising
a) at least one protein from yogurt and
b) one or more substances from the group of plant extracts and/or plant milks and/or vitamins, provitamins and vitamin precursors, and derivatives of vitamins, provitamins and vitamin precursors
   in the weight ratio a):b) 10:1 to 1:4.

The weight ratio of components a) and b) is 7:1 to 1:3, preferably 5:1 to 2:5, more preferably 5:1 to 1:2, particularly preferably 4:1 to 2:3 and in particular 3:1 to 1:1.

In a preferred embodiment of the invention, the surfactant-containing cleansing composition comprises the yogurt protein(s) in an amount of from 0.01 to 5% by weight, preferably 0.02 to 4% by weight and in particular 0.05 to 3% by weight—based on the total weight of the composition.

The yogurt proteins are particularly preferably κ-casein and/or lactophorin.

According to the invention, preference is given to cleansing compositions which comprise plant extracts from green tea, oak bark, stinging nettle, hamamelis, hops, henna, chamomile, burdock, horsetail, hawthorn, linden blossom, almond, aloe vera, spruce needle, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, vanilla, marshmallow, meristem, ginseng and ginger root.

According to the invention, the extracts from green tea, almond, aloe vera, coconut, mango, apricot, lemon, wheat, vanilla, kiwi and melon are particularly preferred, and the extracts from aloe vera, vanilla and melon are especially preferred.

These extracts are usually produced by extraction of the whole plant. However, in individual cases, it may also be preferred to produce the extracts exclusively from flowers and/or leaves of the plant.

Extractants which may be used for producing the specified plant extracts are water, alcohols, and mixtures thereof. Among the alcohols, lower alcohols, such as ethanol and isopropanol, but in particular polyhydric alcohols, such as ethylene glycol and propylene glycol, either as the sole extractant or in a mixture with water, are preferred here. Plant extracts based on water/propylene glycol in the ratio 1:10 to 10:1 have proven to be particularly suitable.

According to the invention, the plant extracts can be used either in pure form or in dilute form. If they are used in dilute form, they comprise about 2-80% by weight of active substance and, as solvent, the extractant or extractant mixture used for their isolation.

The plant extracts are used in the cleansing composition according to the invention—based on its weight—in an amount of from 0.01 to 5% by weight, preferably 0.02 to 4% by weight and in particular 0.05 to 3% by weight.

As plant milks, the compositions according to the invention preferably comprise aloe milk and/or coconut milk, which are used in the surfactant-containing cleansing compositions in an amount of from 0.01 to 5% by weight, preferably 0.02 to 4% by weight and in particular 0.05 to 3% by weight—based on the total weight of the composition.

Vitamins, provitamins and vitamin precursors, and derivatives thereof, preferred according to the invention are to be understood as meaning those representatives which are usually assigned to the groups A, B, C, E, F and H.

The group of the substances referred to as vitamin A includes retinol (vitamin A₁) and 3,4-didehydroretinol (vitamin A₂). β-Carotene is the provitamin of retinol. Suitable as vitamin A component are, according to the invention, for example vitamin A acid and esters thereof, vitamin A aldehyde and vitamin A alcohol, and esters thereof, such as the palmitate and the acetate. The compositions according to the invention comprise the vitamin A component preferably in amounts of 0.01-1% by weight, based on the total preparation.

The vitamin B group or the vitamin B complex includes, inter alia,
vitamin B₁ (thiamine)
vitamin B₂ (riboflavin)
vitamin B₃. This name often covers the compounds nicotinic acid and nicotinamide (niacinamide). According to the invention, preference is given to nicotinamide, which is present in the compositions used according to the invention preferably in amounts of from 0.05 to 1% by weight, based on the total composition.
Vitamin B₅ (pantothenic acid, panthenol and pantolactone). Within the scope of this group, preference is given to using panthenol and/or pantolactone. Derivatives of the panthenol which can be used according to the invention are, in particular, the esters and ethers of panthenol, and cationically derivatized panthenols. Individual representatives are, for example, panthenol triacetate, panthenol monoethyl ether and its monoacetate, and the cationic panthenol derivatives disclosed in WO 92/13829.
Vitamin B₆ (pyridoxine and pyridoxamine and pyridoxal).

The specified compounds of the vitamin B group are present in the compositions according to the invention preferably in amounts of 0.01-2% by weight, based on the total composition. Amounts of 0.03-1% by weight are particularly preferred.

Vitamin C (ascorbic acid). Vitamin C is used in the compositions used according to the invention preferably in amounts of from 0.01 to 3% by weight, based on the total composition. Use in the form of the palmitic acid ester, the glucosides or phosphates may be preferred. Use in combination with tocopherols may likewise be preferred.

Vitamin E (tocopherols, in particular α-tocopherol). Tocopherol and its derivatives, which include, in particular, the esters, such as the acetate, the nicotinate, the phosphate and the succinate, are present in the compositions used according to the invention preferably in amounts of 0.01-1% by weight, based on the total composition.

Vitamin F. The term “vitamin F” is usually understood as meaning essential fatty acids, in particular linoleic acid, linolenic acid and arachidonic acid.

Vitamin H. Vitamin H is the term used to refer to the compound (3aS,4S,6aR)-2-oxohexahydrothienol[3,4-d]imidazole-4-valeric acid, for which, however, the trivial name biotin has meanwhile caught on. Biotin is present in the compositions used according to the invention preferably in amounts of from 0.0001 to 1.0% by weight, in particular in amounts of from 0.001 to 0.01 % by weight.

Preferably, the preparations used according to the invention comprise vitamins, provitamins and vitamin precursors from the groups A, E, F and H. Two or more vitamins and vitamin precursors can of course also be present simultaneously.

The total use amount of the vitamins, provitamins, vitamin precursors, and derivatives thereof, in the compositions according to the invention is—based on the total weight of the composition—0.01 to 5% by weight, preferably 0.02 to 4% by weight and in particular 0.05 to 3% by weight.

The surfactant-containing cleansing composition of the invention comprises—based on its weight—5 to 30% by weight, preferably 7 to 20% by weight and in particular 8 to 14% by weight, of surfactants. These are selected from the group of anionic, amphoteric, zwitterionic and nonionic surfactants, and from mixtures of these surfactant classes.

Suitable anionic surfactants in the preparations according to the invention are all anionic surface-active substances suitable for use on the human body. These are characterized by a water-solubilizing, anionic group such as, for example, a carboxylate group, sulfate group, sulfonate group or phosphate group, and a lipophilic alkyl group having about 8 to 30 carbon atoms. In addition, glycol or polyglycol ether groups, ester groups, ether groups and amide groups, and hydroxyl groups may be present in the molecule. Examples of suitable anionic surfactants are, in each case in the form of the sodium, potassium and ammonium salts, and the mono-, di- and trialkanolammonium salts having 2 to 4 carbon atoms in the alkanol group,
linear and branched fatty acids having 8 to 30 carbon atoms (soaps),
ether carboxylic acids of the formula R—O—(CH₂—CH₂O)ₓ—CH₂—COOH, in which R is a linear alkyl group having 8 to 30 carbon atoms and x is 0 or 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic mono- and dialkyl esters having 8 to 24 carbon atoms in the alkyl group and sulfosuccinic acid monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂O)ₓ—OSO₃H, in which R is a preferably linear alkyl group having 8 to 30 carbon atoms and x is 0 or 1 to 12,
mixtures of surface-active hydroxysulfonates as in DE-A-37 25 030,
sulfated hydroxyalkyl polyethylene and/or hydroxyalkylene propylene glycol ethers as in DE-A-37 23 354,
sulfonates of unsaturated fatty acids having 8 to 24 carbon atoms and 1 to 6 double bonds as in DE-A-39 26 344,
esters of tartaric acid and citric acid with alcohols, which are addition products of about 2-15 molecules of ethylene oxide and/or propylene oxide onto fatty alcohols having 8 to 22 carbon atoms,
alkyl and/or alkenyl ether phosphates of the formula (II) 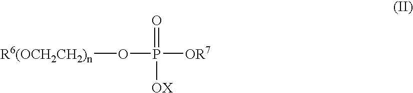
in which R⁶ is preferably an aliphatic hydrocarbon radical having 8 to 30 carbon atoms, R⁷ is hydrogen, a radical (CH₂CH₂O)ₙR⁶ or X, n is numbers from 1 to 10 and X is hydrogen, an alkali metal or alkaline earth metal or NR⁸R⁹R¹⁰R¹¹, where R⁸ to R¹¹, independently of one another, are hydrogen or a C₁ to C₄ hydrocarbon radical,
sulfated fatty acid alkylene glycol esters of the formula (III)
   R¹²CO(AlkO)ₙSO₃M (III)
in which R¹²CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation, as are described in DE-A 197 36 906.5,
monoglyceride sulfates and monoglyceride ether sulfates of the formula (IV), as have been described, for example, in EP-B1 0 561 825, EP-B1 0 561 999, DE-A1 42 04 700 or by A. K. Biswas et al. in J. Am. Oil. Chem. Soc. 37, 171 (1960) and F. U. Ahmed in J. Am. Oil. Chem. Soc. 67, 8 (1990), 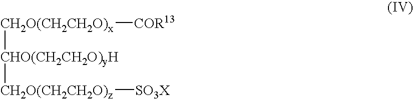
in which R¹³CO is a linear or branched acyl radical having 6 to 22 carbon atoms, x, y and z are in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid in the form of their sodium salts. Preference is given to using monoglyceride sulfates of the formula (VIII) in which R¹³CO is a linear acyl radical having 8 to 18 carbon atoms.

Preferred anionic surfactants are alkyl sulfates, alkyl polyglycol ether sulfates and ether carboxylic acid salts having 10 to 18 carbon atoms in the alkyl group and up to 12 glycol ether groups in the molecule and sulfosuccinic acid mono- and dialkyl esters having 8 to 18 carbon atoms in the alkyl group and sulfosuccinic acid monoalkylpolyoxyethyl esters having 8 to 18 carbon atoms in the alkyl group and 1 to 6 oxyethyl groups. Particularly preferred anionic surfactants are the alkali metal or ammonium salts of lauryl ether sulfate with a degree of ethoxylation of from 2 to 4 EO.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. A preferred zwitterionic surfactant is the fatty acid amide derivative known under the INCI name Cocamidopropyl Betaine.

Ampholytic surfactants are understood as meaning those surface-active compounds which, apart from a C₈-C₂₄-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylamino-propionic acids and alkylaminoacetic acids having in each case about 8 to 24 carbon atoms in the alkyl group. Particularly preferred ampholytic surfactants are N-cocoalkylaminopropionate, cocoacylaminoethylaminopropionate and C₁₂-C₁₈-acylsarcosine.

Nonionic surfactants comprise, as hydrophilic group, e.g., a polyol group, a polyalkylene glycol ether group or a combination of a polyol and a polyglycol ether group. Such compounds are, for example,
addition products of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group,
addition products, terminally capped with a methyl or C₂-C₆-alkyl radical, of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group, such as, for example, the grades available under the brand names Dehydol® LS, Dehydol® LT (Cognis),
C₁₂-C₃₀-fatty acid mono- and diesters of addition products of from 1 to 30 mol of ethylene oxide onto glycerol,
addition products of from 5 to 60 mol of ethylene oxide onto castor oil and hydrogenated castor oil, for example castor oil—hydrogenated+40 EO, as is commercially available, for example, under the trade name Cremophor CO 455 from SHC,
polyol fatty acid esters, such as, for example, the commercial product Hydagen® HSP (Cognis) or Sovermol grades (Cognis),
alkoxylated triglycerides,
alkoxylated fatty acid alkyl esters of the formula (V)
   R¹⁴CO—(OCH₂CHR¹⁵ )_{w}OR¹⁶ (V)
in which R¹⁴CO is a linear or branched, saturated and/or unsaturated acyl radical having 6 to 22 carbon atoms, R¹⁵ is hydrogen or methyl, R¹⁶ is linear or branched alkyl radicals having 1 to 4 carbon atoms and w is numbers from 1 to 20,
amine oxides,
hydroxy mixed ethers, as are described, for example, in DE-A 19738866,
sorbitan fatty acid esters and addition products of ethylene oxide onto sorbitan fatty acid esters, such as, for example, the polysorbates,
sugar fatty acid esters and addition products of ethylene oxide onto sugar fatty acid esters,
addition products of ethylene oxide onto fatty acid alkanolamides and fatty amines,
fatty acid N-alkylglucamides,
alkyl polyglycosides corresponding to the general formula RO-(Z)ₓ, where R is alkyl, Z is sugar, and x is the number of sugar units. The alkyl polyglycosides which can be used according to the invention can contain just one specific alkyl radical R. Usually, however, these compounds are prepared starting from natural fats and oils or mineral oils. In this case, the alkyl radicals R present are mixtures corresponding to the starting compounds or corresponding to the particular work-up of these compounds.

Particular preference is given to those alkyl polyglycosides in which R consists
essentially of C₈- and C₁₀-alkyl groups,
essentially of C₁₂- and C₁₄-alkyl groups,
essentially of C₈- to C₁₆-alkyl groups, or
essentially of C₁₂- to C₁₆-alkyl groups, or
essentially of C₁₆- to C₁₈-alkyl groups.

Sugar building blocks Z which can be used are any monosaccharides or oligosaccharides. Usually, sugars with 5 or 6 carbon atoms, and the corresponding oligosaccharides are used. Such sugars are, for example, glucose, fructose, galactose, arabinose, ribose, silose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides which can be used according to the invention comprise on average 1.1 to 5 sugar units. Alkyl polyglycosides with x values of from 1.1 to 2.0 are preferred. Very particular preference is given to alkyl glycosides in which x is 1.1 to 1.8.

The alkoxylated homologs of the specified alkyl polyglycosides can also be used according to the invention. These homologs can comprise, on average, up to 10 ethylene oxide and/or propylene oxide units per alkyl glycoside unit.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with excellent properties are likewise obtained if they comprise fatty acid esters of ethoxylated glycerol as nonionic surfactants.

These compounds are characterized by the following parameters. The alkyl radical R contains 6 to 22 carbon atoms and may either be linear or branched. Preference is given to primary linear and 2-methyl-branched aliphatic radicals.

Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. When using so-called “oxo alcohols” as starting materials, compounds with an uneven number of carbon atoms in the alkyl chain predominate.

The compounds with alkyl groups used as surfactant may each be single substances. However, it is generally preferred, when producing these substances, to start from native vegetable or animal raw materials, thus giving mixtures of substances with different alkyl chain lengths that are dependent on the respective raw material.

In the case of the surfactants which constitute addition products of ethylene oxide and/or propylene oxide onto fatty alcohols or derivatives of these addition products, it is possible to use either products with a “normal” homolog distribution or those with a narrowed homolog distribution. “Normal” homolog distribution is understood here as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ethercarboxylic acids, alkaline earth metal oxides, hydroxides or alkoxides are used as catalysts. The use of products with a narrowed homolog distribution may be preferred.

According to a further embodiment of the invention, the surfactant-containing cleansing compositions can further comprise cationic surfactants of the quaternary ammonium type, the ester quat type and the amidoamine type.

Preferred quaternary ammonium compounds are ammonium halides, in particular chlorides and bromides, such as alkyltrimethylammonium chlorides, dialkyl-dimethylammonium chlorides and trialkylmethylammonium chlorides, e.g., cetyltri-methylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the above-mentioned surfactants preferably have 10 to 18 carbon atoms.

Ester quats are known substances which contain both at least one ester function and also at least one quaternary ammonium group as structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trade names Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are usually prepared by amidation of natural or synthetic fatty acids and fatty acid cuts with dialkylaminoamines. One compound from this group of substances which is particularly suitable according to the invention is the stearamidopropyldimethylamine commercially available under the name Tegoamid® S 18.

The cationic surfactants are present in the compositions according to the invention preferably in amounts of from 0.05 to 10% by weight, based on the total composition. Amounts of from 0.1 to 5% by weight are particularly preferred.

In a particularly preferred embodiment of the invention, the surface-active cleansing compositions comprise a mixture of at least one anionic surfactant and at least one zwitterionic or amphoteric surfactant. Particular preference is given to a mixture of alkyl sulfates, alkyl ether sulfates and betaines.

In a further preferred embodiment, the effect of the active ingredient according to the invention can be increased through emulsifiers. Such emulsifiers are, for example,
addition products of from 4 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂-fatty acid mono- and diesters of addition products of from 1 to 30 mol of ethylene oxide onto polyols having 3 to 6 carbon atoms, in particular onto glycerol,
ethylene oxide and polyglycerol addition products onto methyl glucoside fatty acid esters, fatty acid alkanolamides and fatty acid glucamides,
C₈-C₂₂-alkyl mono- and oligoglycosides and ethoxylated analogs thereof, where degrees of oligomerization of from 1.1 to 5, in particular 1.2 to 2.0, and glucose as sugar component are preferred,
mixtures of alkyl (oligo)glucosides and fatty alcohols, for example the commercially available product Montanov®68,
addition products of from 5 to 60 mol of ethylene oxide onto castor oil and hydrogenated castor oil,
partial esters of polyols having 3 to 6 carbon atoms with saturated fatty acids having 8 to 22 carbon atoms,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. This is understood in particular as meaning the glucose phospholipids, which are obtained, for example, as lecithins or phosphatidylcholines from e.g., egg yolk or plant seeds (e.g., soya beans).
fatty acid esters of sugars and sugar alcohols, such as sorbitol,
polyglycerols and polyglycerol derivatives, such as, for example, polyglycerol poly-12-hydroxystearate (commercial product Dehymuls® PGPH),
linear and branched fatty acids having 8 to 30 carbon atoms and the Na, K, ammonium, Ca, Mg and Zn salts thereof.

The compositions according to the invention comprise the emulsifiers preferably in amounts of 0.1-25% by weight, in particular 0.5-15% by weight, based on the total composition.

Preferably, the compositions according to the invention can comprise at least one nonionogenic emulsifier with an HLB value of from 8 to 18, according to the definitions listed in Römp Lexikon Chemie (ed. J. Falbe, M. Regitz), 10th edition, Georg Thieme Verlag Stuttgart, New York (1997), page 1764. Nonionogenic emulsifiers with an HLB value of a 10-15 may be particularly preferred according to the invention.

In a particularly preferred embodiment of the invention, the surfactant-containing cleansing compositions further comprise one or more skin- and/or hair-conditioning substance. According to the invention, these are selected from cationic polymers, oil bodies and/or protein hydrolyzates.

Cationic polymers are to be understood as meaning polymers which, in the main chain and/or side chain, have groups which may be “temporarily” or “permanently” cationic. According to the invention, the term “permanently cationic” is used to refer to those polymers which have a cationic group irrespective of the pH of the composition. These are generally polymers which contain a quaternary nitrogen atom, for example in the form of an ammonium group. Preferred cationic groups are quaternary ammonium groups. In particular, those polymers in which the quaternary ammonium group is bonded by means of a C1-4 hydrocarbon group to a polymer main chain constructed from acrylic acid, methacrylic acid or derivatives thereof have proven to be particularly suitable.

Homopolymers of the general formula (IV) 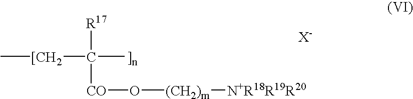
in which R¹⁷═—H or —CH₃, R¹⁸, R¹⁹ and R²⁰, independently of one another, are chosen from C1-4-alkyl, -alkenyl or -hydroxyalkyl groups, m=1, 2, 3 or 4, n is a natural number and X⁻ is a physiologically compatible organic or inorganic anion, and copolymers consisting essentially of the monomer units listed in formula (III), and nonionogenic monomer units, are particularly preferred cationic polymers. Among these polymers, preference is given according to the invention to those for which at least one of the following conditions applies:
R¹⁷ is a methyl group
R¹⁸, R¹⁹ and R²⁰ are methyl groups
m has the value 2.

Suitable physiologically compatible counterions X⁻ are, for example, halide ions, sulfate ions, phosphate ions, methosulfate ions, and organic ions, such as lactate, citrate, tartrate and acetate ions. Preference is given to halide ions, in particular chloride.

A suitable homopolymer is, if desired crosslinked, poly(methacryloyloxyethyltrimethylammonium chloride) with the INCI name Polyquaternium-37. The crosslinking can take place if desired with the help of polyolefinically unsaturated compounds, for example divinylbenzene, tetraallyloxyethane, methylenebisacrylamide, diallyl ether, polyallyl polyglyceryl ether, or allyl ethers of sugars or sugar derivatives, such as erythritol, pentaerythritol, arabitol, mannitol, sorbitol, sucrose or glucose. Methylenebisacrylamide is a preferred crosslinking composition.

The homopolymer is preferably used in the form of a nonaqueous polymer dispersion which should have a polymer fraction not below 30% by weight. Such polymer dispersions are commercially available under the names Salcare® SC 95 (about 50% polymer fraction, further components: mineral oil (INCI name: Mineral Oil) and tridecyl polyoxypropylene polyoxyethylene ether (INCI name: PPG-1-Trideceth-6)) and Salcare® SC 96 (about 50% polymer fraction, further components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (VI) comprise, as nonionogenic monomer units, preferably acrylamide, methacrylamide, C₁₋₄-alkyl acrylates and C₁₋₄-alkyl methacrylates. Among these nonionogenic monomers, particular preference is given to acrylamide. As in the case of the homopolymers described above, these copolymers too may be crosslinked. A copolymer preferred according to the invention is the crosslinked acrylamide-methacryloyloxethyltrimethylammonium chloride copolymer. Such copolymers in which the monomers are present in a weight ratio of about 20:80 are commercially available as about 50% strength nonaqueous polymer dispersion under the name Salcare® SC 92.

Further preferred cationic polymers are, for example,
quaternized cellulose derivatives, as are commercially available under the names Celquat® and Polymer JR®. The compounds Celquat® H 100, Celquat® L 200 and Polymer JR®400 are preferred quaternized cellulose derivatives,
cationic alkyl polyglycosides as in DE-C 44 13 686,
cationized honey, for example the commercial product Honeyquat® 50,
cationic guar derivatives, such as, in particular, the products sold under the trade names Cosmedia® Guar and Jaguar®,
polysiloxanes with quaternary groups, such as, for example, the commercially available products Q2-7224 (manufacturer: Dow Corning; a stabilized trimethylsilylamodimethicone), Dow Corning® 929 emulsion (comprising a hydroxylamino-modified silicone, which is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80),
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are supplied under the names Luviquat® FC 370, FC 550, FC 905 and HM 552,
quaternized polyvinyl alcohol,
   and the polymers known under the names
Polyquaternium 2,
Polyquaternium 17,
Polyquaternium 18 and
Polyquaternium 27 with quaternary nitrogen atoms in the polymer main chain.

As cationic polymers it is likewise possible to use the polymers known under the names Polyquaternium-24 (commercial product, e.g., Quatrisoft® LM 200). According to the invention, it is likewise possible to use the copolymers of vinylpyrrolidone, as are obtainable as commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Further cationic polymers according to the invention are the so-called “temporarily cationic” polymers. These polymers usually comprise an amino group which is present as quaternary ammonium group and thus in cationic form at certain pH values. Preference is given, for example, to chitosan and derivatives thereof, as are freely available commercially, for example, under the trade names Hydagen® CMF, Hydagen® HCMF, Kytamer® PC and Chitolam® NB/101. Chitosans are deacetylated chitins which are commercially available in varying degrees of deacetylation and varying degrees of degradation (molecular weights). Their preparation is described, for example, in DE 44 40 625 A1 and in DE 1 95 03 465 A1.

Particularly highly suitable chitosans have a degree of deacetylation of at least 80% and a molecular weight of 5·10⁵ to 5·10⁶ (g/mol).

For the production of the preparations according to the invention, the chitosan must be converted to the salt form. This can take place through dissolution in dilute aqueous acids. Suitable acids are either mineral acids, such as, for example, hydrochloric acid, sulfuric acid and phosphoric acid, or organic acids, for example lower molecular weight carboxylic acids, polycarboxylic acids and hydroxycarboxylic acids. It is also possible to use higher molecular weight alkylsulfonic acids or alkylsulfuric acids or organophosphoric acids provided these have the required physiological compatibility. Suitable acids for converting the chitosan into the salt form are, for example, acetic acid, glycolic acid, tartaric acid, malic acid, citric acid, lactic acid, 2-pyrrolidinone-5-carboxylic acid, benzoic acid or salicylic acid. Preference is given to using lower molecular weight hydroxycarboxylic acids, such as, for example, glycolic acid or lactic acid.

In a particularly preferred embodiment of the invention, the cationic polymer present in the cleansing compositions is at least one polymer from the group Polyquaternium-7 (Merquat 550), Polyquaternium-6 or Polyquaternium-10.

The cationic polymers are present in the compositions according to the invention preferably in amounts of from 0.1 to 5% by weight, based on the total composition. Amounts of from 0.2 to 3% by weight, in particular from 0.5 to 2% by weight, are particularly preferred.

According to the invention, vegetable, mineral or synthetic oils, and mixtures of these components, can be used in the cleansing compositions according to the invention as water-insoluble oil component.

The natural (vegetable) oils used are usually triglycerides and mixtures of triglycerides. Preferred natural oils for the purposes of the invention are coconut oil, (sweet) almond oil, walnut oil, peach kernel oil, avocado oil, tea tree oil, soybean oil, sesame oil, sunflower oil, tsubaki oil, evening primrose oil, rice bran oil, palm kernel oil, mango kernel oil, lady's smock oil, thistle oil, macadamia nut oil, grapeseed oil, apricot kernel oil, babusu oil, olive oil, wheat germ oil, pumpkin seed oil, mallow oil, hazelnut oil, safflower oil, canola oil, sasanqua oil, jojoba oil and shea butter.

The mineral oils used are, in particular, mineral oils, paraffin and isoparaffin oils, and synthetic hydrocarbons. A hydrocarbon which can be used according to the invention is, for example, the commercially available product 1,3-di(2-ethylhexyl)cyclohexane (Cetiol® S).

Suitable synthetic oils are silicone compounds, in particular dialkyl- and alkylarylsilicones, such as, for example, dimethylpolysiloxane and methylphenylpolysiloxane, and their hydroxy-terminated, alkoxylated and quaternized analogs. Examples of such silicones are the products sold by Dow Corning under the names DC 190, DC 200, DC 344 and DC 345 (cyclomethicones).

Silicone compounds preferred according to the invention are linear and cyclic, nonalkoxylated dialkylsiloxanes and alkylarylsiloxanes.

Furthermore, a dialkyl ether can serve as oil component.

Dialkyl ethers which can be used according to the invention are, in particular, di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, and di-tert-butyl ether, di-isopentyl ether, di-3-ethyldecyl ether, tert-butyl n-octyl ether, isopentyl n-octyl ether and 2-methylpentyl n-octyl ether.

According to the invention, particular preference is given to the di-n-octyl ether which is commercially available under the name Cetiol® OE.

The cleansing compositions according to the invention comprise the water-insoluble oil component preferably in a quantitative range from 0.1 to 5% by weight, in particular from 0.5 to 2% by weight, based on the total weight of the composition.

In a further preferred embodiment of the invention, the effect of the active ingredient combination according to the invention can be further optimized through further fatty substances. Further fatty substances are to be understood as meaning fatty acids, fatty alcohols, and natural and synthetic waxes, which may be either in solid form or in liquid form in aqueous dispersion.

Fatty acids which can be used are linear and/or branched, saturated and/or unsaturated fatty acids having 6-30 carbon atoms. Preference is given to fatty acids having 10-22 carbon atoms. For mention among these would, for example, be the isostearic acids, such as the commercial products Emersol® 871 and Emersol® 875, and isopalmitic acids, such as the commercial product Edenor® IP 95, and all other fatty acids sold under the trade names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid, and technical-grade mixtures thereof which are produced, for example, during the pressurized cleavage of natural fats and oils, during the oxidation of aldehydes from the Roelen oxo synthesis or the dimerization of unsaturated fatty acids. Of particular preference are usually the fatty acid cuts which are obtainable from coconut oil or palm oil; as a rule, the use of stearic acid is particularly preferred.

The use amount here is 0.1-15% by weight, based on the total composition. In a preferred embodiment, the amount is 0.5-10% by weight, with amounts of 1-5% by weight being very particularly advantageous.

Fatty alcohols which can be used are saturated, mono- or polyunsaturated, branched or unbranched fatty alcohols having C₆-C₃₀, preferably C₁₀-C₂₂ and very particularly preferably C₁₂-C₂₂ carbon atoms. For the purposes of the invention, it is possible to use, for example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and Guerbet alcohols thereof, the intention for this list to be exemplary and nonlimiting in character. However, the fatty alcohols originate from preferably natural fatty acids, where the starting point may usually be isolation from the esters of the fatty acids by reduction. According to the invention, it is likewise possible to use those fatty alcohol cuts which are produced by reduction of naturally occurring triglycerides such as beef tallow, palm oil, peanut oil, rapeseed oil, cottonseed oil, soybean oil, sunflower oil and linseed oil or fatty acid esters which form from their transesterification products with corresponding alcohols, and thus constitute a mixture of different fatty alcohols. Such substances are commercially available, for example, under the names Stenol®, e.g., Stenol® 1618, or Lanette®, e.g., Lanette® O, or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. According to the invention, it is of course also possible to use wool wax alcohols, as are commercially available, for example, under the names Corona®, White Swan®, Coronet® or Fluilan®.

The fatty alcohols are used in amounts of 0.1-20% by weight, based on the total preparation, preferably in amounts of 0.1-10% by weight.

Natural or synthetic waxes which can be used according to the invention are solid paraffins or isoparaffins, carnauba waxes, beeswaxes, candelilla waxes, ozokerite, ceresin, spermaceti, sunflower wax, fruit waxes, such as, for example, apple wax or citrus wax, microwaxes of PE or PP. Waxes of this type are available, for example, from Kahl & Co., Trittau.

Further fatty substances are, for example,
Ester oils. Ester oils are to be understood as meaning the esters of C₆-C₃₀-fatty acids with C₂-C₃₀-fatty alcohols. Preference is given to the monoesters of the fatty acids with alcohols having 2 to 24 carbon atoms. Examples of fatty acid fractions used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid, and technical-grade mixtures thereof which are produced, for example, during the pressurized cleavage of natural fats and oils, during the oxidation of aldehydes from the Roelen oxo synthesis or the dimerization of unsaturated fatty acids. Examples of the fatty alcohol fractions in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof which are produced, for example, during the high-pressure hydrogenation of technical-grade methyl esters based on fats and oils or aldehydes from the Roelen oxo synthesis, and as monomer fraction during the dimerization of unsaturated fatty alcohols. According to the invention, particular preference is given to isopropyl myristate (Rilanit® IPM), isononanoic acid C16-C18-alkyl ester (Cetiol® SN), 2-ethylhexyl palmitate (Cegesoft® 24), 2-ethylhexyl stearate (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), hexyl laurate (Cetiol® A), di-n-butyl adipate (Cetiol® B), myristyl myristate (Cetiol® MM), cetearyl isononanoate (Cetiol® SN), decyl oleate (Cetiol® V).
Dicarboxylic acid esters, such as di-n-butyl adipate, di(2-ethylhexyl) adipate, di(2-ethylhexyl)succinate and diisotridecyl azelate, and diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,
symmetric, asymmetric or cyclic esters of carbon acid with fatty alcohols, described for example in DE-A 197 56 454, glycerol carbonate or dicaprylyl carbonate (Cetiol® CC),
ethoxylated or nonethoxylated mono-, di- and trifatty acid esters of saturated and/or unsaturated linear and/or branched fatty acids with glycerol, such as, for example, Monomuls® 90-018, Monomuls® 90-L12 or Cutina® MD.

The use amount is 0.1-50% by weight, based on the total composition, preferably 0.1-20% by weight and particularly preferably 0.1-15% by weight, based on the total composition.

The total amount of oil and fat components in the compositions according to the invention is usually 6-45% by weight, based on the total composition. Amounts of 10-35% by weight are preferred according to the invention.

In a further preferred embodiment of the invention, the cleansing compositions can further comprise one or more representatives from the group of protein hydrolyzates or of pearlescent substances.

For the purposes of the invention, protein hydrolyzates are understood as meaning protein hydrolyzates and/or amino acids and derivatives thereof (H). Protein hydrolyzates are product mixtures which are obtained by acid-, base- or enzyme-catalyzed degradation of proteins. According to the invention, the term protein hydrolyzates is also understood as meaning total hydrolyzates, and individual amino acids and derivatives thereof, and mixtures of different amino acids. According to the invention, polymers constructed from amino acids and amino acid derivatives are also understood under the term protein hydrolyzates. The latter include, for example, polyalanine, polyasparagine, polyserine etc. Further examples of compounds that can be used according to the invention are L-alanyl-L-proline, polyglycine, glycyl-L-glutamine or D/L-methionine-S-methylsulfonium chloride. According to the invention, it is, of course, also possible to use β-amino acids and derivatives thereof, such as β-alanine, anthranilic acid or hippuric acid. The molecular weight of the protein hydrolyzates that can be used according to the invention is between 75, the molecular weight of glycine, and 200,000. Preferably the molecular weight is 75 to 50,000 and very particularly preferably, 75 to 20,000 daltons.

According to the invention, protein hydrolyzates either of vegetable origin or of animal or marine or synthetic origin can be used.

Animal protein hydrolyzates are, for example, elastin, collagen, keratin, silk and milk protein hydrolyzates, which may also be in the form of salts. Such products are sold, for example, under the trade names Dehylan® (Cognis), Promois® (Interorgana), Callapuron® (Cognis), Nutrilan® (Cognis), Gelita-Sol® (Deutsche Gelatine Fabriken Stoess & Co), Lexein® (Inolex) and Kerasol® (Croda).

According to the invention, the use of protein hydrolyzates of vegetable origin is preferred, e.g., soybean, almond, pea, potato and wheat protein hydrolyzates. Such products are available, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although the use of protein hydrolyzates as such is preferred, instead of them, it is also possible to use, if appropriate, amino acid mixtures obtained in another way. The use of derivatives of protein hydrolyzates is likewise possible, for example in the form of their fatty acid condensation products. Such products are sold, for example, under the names Lamepon® (Cognis), Lexein® (Inolex), Crolastin® (Croda) or Crotein® (Croda).

The protein hydrolyzates or derivatives thereof are present in the preparations used according to the invention preferably in amounts of from 0.1 to 10% by weight, based on the total composition. Amounts of from 0.1 to 5% by weight are particularly preferred.

Further active ingredients, auxiliaries and additives are, for example,
thickeners, such as agar agar, guar gum, alginates, xanthan gum, gum arabic, karaya gum, carob seed flour, linseed gums, dextrans, cellulose derivatives, e.g., methylcellulose, hydroxyalkylcellulose and carboxymethylcellulose, starch fractions and derivatives, such as amylose, amylopectin and dextrins, clays, such as, for example, bentonite or completely synthetic hydrocolloids such as, for example, polyvinyl alcohol,
hair-conditioning compounds, such as phospholipids, for example soybean lecithin, egg lecithin and cephalins, and silicone oils,
perfume oils, dimethyl isosorbide and cyclodextrins,
solvents and solubility promoters, such as ethanol, isopropanol, ethylene glycol, propylene glycol, glycerol and diethylene glycol,
active ingredients which improve fiber structure, in particular mono-, di- and oligosaccharides, such as, for example, glucose, galactose, fructose, fruit sugar and lactose,
antifoams, such as silicones,
dyes for coloring the composition,
antidandruff active ingredients such as piroctone olamine, zinc omadine and climbazole,
active ingredients, such as allantoin and bisabolol, cholesterol,
consistency regulators, such as sugar esters, polyol esters or polyol alkyl ethers,
fats and waxes, such as spermaceti, beeswax, montan wax and paraffins,
fatty acid alkanolamides,
complexing agents, such as EDTA, NTA, β-alaninediacetic acid and phosphonic acids,
swelling and penetration substances, such as primary, secondary and tertiary phosphates,
opacifiers, such as latex, styrene/PVP and styrene/acrylamide copolymers,
pearlizing agents, such as ethylene glycol mono- and distearate, and PEG-3 distearate,
pigments,
propellants, such as propane/butane mixtures, N₂O, dimethyl ether, CO₂ and air,
antioxidants.

With regard to further optional components and the amounts used of these components, reference is expressly made to the relevant handbooks known to the person skilled in the art, e.g., the monograph by K. H. Schrader, Grundlagen und Rezepturen der Kosmetika [Fundamentals and formulations of cosmetics], 2nd edition, Hüthig Buch Verlag Heidelberg, 1989.

With regard to their formulation form, the cleansing compositions according to the invention are not subject to any limitations of any kind and can be formulated as emulsion, cream, solution, gel or mousse.

The invention also provides a method for smoothing the skin comprising applying an effective amount of a surfactant-containing cleansing composition comprising a mixture of yogurt proteins and plant extracts and/or plant milks and/or vitamins, provitamins and vitamin and provitamin precursors.

The invention also provides a method for regulating skin moisture comprising applying an effective amount of a surfactant-containing cleansing composition comprising a mixture of yogurt proteins and plant extracts and/or plant milks and/or vitamins, provitamins and vitamin and provitamin precursors.

### EXAMPLES

The following shower baths according to the invention were prepared:

| | A | B | C | D | E |
|---|---|---|---|---|---|
| | % | % | % | % | % |
| | telquel | telquel | telquel | telquel | telquel |
| Texapon ®¹ N 70 | 12 | 10 | 8 | 10 | 14 |
| Dehyton ®² PS | 9 | | 4 | 10 | 2 |
| Rewotoric ®³ AMC | | 7 | 6 | | |
| Plantacare ®⁴ 818 | 2 | 1.5 | | | 5 |
| Conditioner ®⁵ P7 | 1 | 0.5 | | 2 | |
| Panthenol | 0.2 | | 0.1 | | |
| Eupertan ®⁶ PK 810 | 0.5 | | | 2 | 2 |
| Acusol ®⁷ OP 301 | 2 | | 3 | 0.5 | |
| Vitamin E | | 0.1 | 0.2 | | |
| Vitamin complex | 0.2 | | | | |
| A, E, F, H | | | | | |
| Yogurt protein | 0.5 | 0.2 | 0.1 | 0.3 | 0.4 |
| Aloe vera extract | | | | | 0.1 |
| Vanilla extract | | | | 0.1 | |
| Melon extract | | 0.3 | | | |
| Aloe vera milk | | | 0.2 | | |
| Coconut milk | 0.2 | | | | |
| Water | ad 100 | ad 100 | ad 100 | ad 100 | ad 100 |

¹sodium lauryl ether sulfate, 2 EO (INCI: SODIUM LAURETH SULFATE); AS: 68%; Cognis
²coconut fatty acid amidoethyl N-2-hydroxyethylglycine sodium salt (INCI: DISODIUM COCOAMPHODIACETATE); AS: 37.5%; Pulcra Iberica
³hydroxyethyl N-cocoalkylamidoethyl carboxymethylglycinate sodium salt (INCI: SODIUM COCOAMPHOACETATE); AS: 30.5-32%; Degussa
⁴C₈-C₁₄-alkyl polyglucoside (INCI: COCO-GLUCOSIDE); AS: 51-53%; Cognis
⁵acrylamide dimethyldiallylammonium chloride copolymer (INCI: POLYQUATERNIUM-7); 3V Sigma
⁶pearlizing agent (INCI: AQUA; GLYCOL DISTEARATE; SODIUM LAURETH SULFATE; COCAMIDE MEA; LAURETH-10, FORMIC ACID); Cognis
⁷styrene acrylate copolymer (INCI: STYRENE/ACRYLATES COPOLYMER); AS: 39-41%; Rohm & Haas

1. A surfactant-containing cleansing composition comprising a surfactant and
a) at least one yogurt protein and
b) one or more substances selected from the group consisting of plant extracts, plant milks, vitamins and derivatives thereof, provitamins and derivatives thereof, vitamin precursors, and derivatives thereof, and combinations thereof
wherein the weight ratio a):b) is from 10:1 to 1:4.

2. The surfactant-containing cleansing composition of claim 1 wherein the weight ratio of a):b) is from 7:1 to 1:3.

3. The surfactant-containing cleansing composition of claim 2 wherein the weight ratio of a):b) is from 3:1 to 1:1.

4. The surfactant-containing cleansing composition of claim 1 wherein the plant extracts are selected from the group consisting of aloe extract, melon extract, and vanilla extract.

5. The surfactant-containing cleansing composition of claim 1 wherein the plant milks are selected from the group consisting of aloe milk and coconut milk.

6. The surfactant-containing cleansing composition of claim 1 wherein the vitamins, provitamins and vitamin precursors are vitamins A, B, C, E, F and H.

7. The surfactant-containing cleansing composition of claim 1 wherein the amount of the yogurt protein is from 0.01 to 5% by weight.

8. The surfactant-containing cleansing composition of claim 7 wherein the amount of the yogurt protein is from 0.05 to 3% by weight.

9. The surfactant-containing cleansing composition of claim 1 wherein the amount of component b) is from 0.01 to 5% by weight.

10. The surfactant-containing cleansing composition of claim 7 wherein the amount of component b) is from 0.05 to 3% by weight.

11. The surfactant-containing cleansing composition of claim 1 wherein the yogurt protein is κ-casein, lactophorin or a combination thereof.

12. The surfactant-containing cleansing composition of claim 1 wherein the surfactant is an anionic surfactant, amphoteric surfactant, zwitterionic surfactant, nonionic surfactant, or a mixture thereof.

13. The surfactant-containing cleansing composition of claim 12 wherein the amount of the surfactant is from 5 to 30% by weight.

14. The surfactant-containing cleansing composition of claim 13 wherein the amount of the surfactant is from 8 to 14% by weight.

15. A method for smoothing the skin comprising applying an effective amount of a surfactant-containing cleansing composition comprising a mixture of yogurt proteins and plant extracts and/or plant milks and/or vitamins, provitamins and vitamin and provitamin precursors.

16. A method for regulating skin moisture comprising applying an effective amount of a surfactant-containing cleansing composition comprising a mixture of yogurt proteins and plant extracts and/or plant milks and/or vitamins, provitamins and vitamin and provitamin precursors.
